(19) United States
(12) Patent Application Publication
(10) Pub. No.: US 2005/0176079 A1
(43) Pub. Date: Aug. 11, 2005

(54) Polypeptide bioconjugates, methods of making the bioconjugates and assays employing the bioconjugates

(76) Inventors: Yong Liang Chu, Potomac, MD (US); Wan-Ching Lai, Rockville, MD (US); Frank Q. Li, Montgomery Village, MD (US)

Correspondence Address: Supervisor, Patent Prosecution Services;PIPER RUDNICK LLP, 1200 Nineteenth Street, N.W., Washington, DC, 20036-2412, US

(21) Appl. No.: 10/773,274

(22) Filed: Feb. 9, 2004

(51) Int. Cl. G01N 33/53; G01N 33/537; G01N 33/543

(52) U.S. Cl. 435/7.92

(57) ABSTRACT

A method for detecting the presence or amount of an analyte in a sample is described. The method involves contacting the surface of the solid support with the sample and detecting analyte on the surface of the solid support. A bioconjugate comprising a polypeptide conjugated to a polysaccharide is deposited on the surface of the solid support and the polypeptide of the bioconjugate can interact with the analyte. The analyte can be an antibody and the polypeptide can comprise an antigenic determinant of the antibody. By conjugating the polypeptide to the polysaccharide, the antibody can be detected at lower levels than if the non-conjugated polypeptide were employed. Exemplary solid supports include membranes and assay plates (e.g., ELISA plates). The polysaccharide is preferably a hyaluronic acid or a hyaluronic acid analogue. The method can be used to diagnose diseases such as SARS. Methods of making polypeptide-polysaccharide bioconjugates are also provided.

This application is related to U.S. patent application Ser. No. 10/062,710, filed Feb. 5, 2002. This application is also related to provisional U.S. Patent Application Ser. No. 60/475,486, filed Jun. 4, 2003 and provisional U.S. Patent Application Ser. No. 60/524,614, filed Nov. 25, 2003. Each of the aforementioned applications is incorporated herein by reference in its entirety.

### BACKGROUND

1. Technical Field

The present application relates generally to bioconjugates of polypeptides and, in particular, to: bioconjugates of polypeptides and polysaccharides; methods of making the bioconjugates; and methods of using the bioconjugates in assays.

2. Background of the Technology

Antibodies are proteins generated by animals in response to the invasion of a foreign molecule (i.e., an antigen) into the body. Antibodies are found in blood and tissue fluids and can bind to the antigen when it is encountered. Because antibodies are developed based on the specific three-dimensional structure of an antigen, they are highly specific and will bind only to that structure.

In an antigen-down immunoassay, an antigen is coated onto a solid support surface and used to bind antibodies found in a sample. When the sample is added (e.g., human serum), the antigen on the plate can be bound by antibodies (e.g., IgE, IgG, IgA, or IgM) from the sample. An enzyme or fluorescent labeled species-specific antibody (e.g., anti-human IgE) can then be added which binds to the antibody bound to the antigen on the solid support. The presence of a signal on the solid support indicates the presence of the antibody in the sample.

Severe Acute Respiratory Syndrome (SARS) is a respiratory disorder of humans where patients exhibit both flu-like and cold-like symptoms. Although the disease was originally identified in China and various other portions of South East Asia, it has been exported by human travel and contact to North and South America, Europe, Africa and parts of the Middle East. The causative virus has been identified as a corona virus, the genome and encoded protein structure having been deduced and posted no later than Apr. 12, 2003. The genome is 29,736 nucleotides long. Other versions of the genome have been published with trivial differences.

Notwithstanding the focused attention of a vast amount of medical and biological resources on the problem, the detection, treatment and prevention of SARS remain pressing concerns that have not been addressed. For example, the current method of detection of SARS is PCR analysis. While reliable, PCR is a laboratory assay that requires a substantial investment in apparatus and infrastructure.

Accordingly, there still exists a need for improved methods for detecting antibodies to diseases such as SARS having higher sensitivities which would allow for the detection of the antibodies at relatively low concentrations.

### SUMMARY OF THE INVENTION

According to a first embodiment of the invention, a method for detecting the presence or amount of an analyte in a sample is provided. The method comprises contacting a surface of a solid support with the sample and detecting analyte on the surface of the solid support. According to this embodiment, the surface of the solid support comprises a conjugate of a polypeptide and a polysaccharide wherein the polypeptide of the bioconjugate is capable of interacting with the analyte. According to a further embodiment, the polypeptide of the bioconjugate is capable of binding to the analyte. The analyte can be an antibody, an immunoglobulin, an enzyme, or a hormone. For example, the analyte can be an antibody and the polypeptide can comprise an antigenic determinant of the antibody. By conjugating the polypeptide comprising an antigenic determinant of the antibody to the polysaccharide, the antibody can be detected at lower levels than if the non-conjugated polypeptide were employed. Exemplary solid supports include, but are not limited to, membranes and assay plates (e.g., ELISA plates). The polysaccharide is preferably a hyaluronic acid or a hyaluronic acid analogue.

Detecting can comprise contacting the surface of the solid support with a secondary antibody which binds to the analyte in the sample and detecting the secondary antibody. The secondary antibody can be labeled (e.g., with an enzyme or fluorescent label). A wash step can be conducted after contact of the solid support surface with the sample and before contact with the secondary antibody. Wash steps can also be conducted after contact of the solid support surface with the secondary antibody and before detection and/or after depositing the bioconjugate on the solid support and before contact with the sample.

According to a second embodiment of the invention, a method of making a polypeptide bioconjugate is provided wherein a polypeptide having a free amino group is covalently linked to a polysaccharide. According to this aspect of the invention, the polysaccharide is reacted with a first linker compound having at least two carboxylic acid reactive groups (e.g., a diamine) such that one carboxylic acid reactive group of the first linker compound reacts with a carboxylic acid group of the polysaccharide. The resulting reaction product is then reacted with an acid anhydride or a dicarboxylic acid such that an anhydride group of the acid anhydride or a carboxylic acid group of the dicarboxylic acid reacts with the remaining carboxylic acid reactive group of the first linker compound. The polypeptide comprising a free amino group is then reacted with the resulting reaction product such that the free amino group reacts with the remaining carboxylic acid group of the dicarboxylic acid to form the polypeptide bioconjugate. Exemplary first linker compounds include diamines (e.g., alkane diamines such as diaminoethane). Exemplary acid anhydrides include, but are not limited to, succinic anhydride and glutaric anhydride. Exemplary dicarboxylic acids include, but are not limited to, succinic acid, glutaric acid and adipic acid. Each step in the reaction set forth above can be conducted under weak acidic conditions (e.g., at a pH of about 5) and in the presence of EDC. Bioconjugates made by a method as set forth above are also provided. Solid supports comprising a bioconjugate as set forth above disposed on a surface thereof are also provided. Exemplary solid supports include, but are not limited to, membranes and assay plates (e.g., nitrocellulose paper, nylon, or ELISA plates).

According to a third embodiment of the invention, a method of making a polypeptide bioconjugate is provided wherein a polypeptide having a free sulfhydryl (i.e., cystein) group is covalently linked to a polysaccharide. According to this aspect of the invention, the polysaccharide is reacted with a dihydrazide such that a hydrazide group of the dihydrazide reacts with a carboxylic acid group of the polysaccharide. This step in the reaction can be conducted under weak acidic conditions (e.g., at a pH of about 5) in the presence of EDC. The resulting reaction product is then reacted with a linker compound comprising an amino reactive functional group and a thiol reactive functional group (e.g., sulfo-LC-SMPT) such that the amino reactive group of the linker compound reacts with the remaining hydrazide group of the dihydrazide. The polypeptide comprising a sulfhydryl group is then reacted with the resulting reaction product such that the free sulfhydryl group reacts with the remaining thiol reactive group of the linker compound residue to form the polypeptide bioconjugate. Exemplary dihydrazides include, but are not limited to, adipic dihydrazide. Exemplary linker compounds comprising an amino reactive functional group and a thiol reactive functional group include, but are not limited to, sulfo-LC-SMPT. Bioconjugates made by a method as set forth above are also provided. Solid supports comprising a bioconjugate as set forth above disposed on a surface thereof are also provided. Exemplary solid supports include, but are not limited to, membranes and assay plates (e.g., nitrocellulose paper, nylon, or ELISA plates).

According to a fourth embodiment of the invention, a bioconjugate is provided comprising a polypeptide residue conjugated to a polysaccharide residue by a linker moiety wherein the linker moiety comprises the residue of: a diamino compound; a dihydrazide compound; a diamino and a dicarboxylic acid compound; or a dihydrazide and a compound comprising an amino reactive functional group and a thiol reactive functional group. Solid supports comprising a bioconjugate as set forth above disposed on a surface thereof are also provided. Exemplary solid supports include, but are not limited to, membranes and assay plates (e.g., ELISA plates).

### BRIEF DESCRIPTION OF THE DRAWINGS

FIG. 1 illustrates the synthesis of a polypeptide bioconjugate according to a second embodiment of the invention wherein the polypeptide comprises a free amino group.

FIG. 2 illustrates the synthesis of a polypeptide bioconjugate according to a third embodiment of the invention wherein the polypeptide comprises a free sulfhydryl group.

FIG. 3 is an image of a membrane having various concentrations of a polypeptide (e.g., “peptide T”) or a polypeptide-polysaccharide bioconjugate deposited thereon wherein the membrane has been assayed for the presence of the polypeptide.

FIG. 4 is a chart showing the effect of the concentration of a hyaluronate peptide T conjugate on ELISA detection limits.

### DETAILED DESCRIPTION

Methods for determining the presence and/or amount of an antibody which binds to an antigenic determinant of a polypeptide in a sample are provided. According to one embodiment, the method comprises: covalently linking a polypeptide comprising the antigenic determinant to a polysaccharide to form a bioconjugate; depositing the bioconjugate on a surface of the solid support; contacting the surface of the solid support with the sample to allow antibody in the sample to bind to the bioconjugate on the solid support; and detecting the antibody on the solid support. Detecting can comprise contacting the surface of the solid support with a secondary antibody which binds to the antibody in the sample and detecting the secondary antibody. The secondary antibody can be labeled (e.g., with an enzyme or fluorescent label). According to this aspect of the invention, by conjugating the polypeptide comprising the antigenic determinant to the polysaccharide, the antibody which binds to the antigenic determinant can be detected in the sample at lower levels than if the non-conjugated polypeptide were used.

Exemplary solid supports include, but are not limited to, membranes and assay plates (e.g., ELISA plates). The solid support surface can comprise nitrocellulose or nylon. For example, the solid support can be a nitrocellulose or a nylon assay paper. The method can allow for detection of amounts of the polypeptide of 10⁻² ng or less.

The method can further comprise one or more wash steps wherein the surface of the solid support is washed. For example, a wash step can be conducted after contact of the solid support surface with the sample and before contact with the secondary antibody. A wash step can also be conducted after contact with the secondary antibody and before detection. Further, a wash step can be conducted after depositing the bioconjugate on the solid support and before contact with the sample.

The secondary antibody can comprise a label such as an emzyme label. Exemplary enzyme labels include, but are not limited to, alkaline phosphatase and horseradish peroxidase. After the secondary antibody is contacted with the surface of the solid support, a substrate for the enzyme can then be contacted with the solid support surface wherein the substrate, when cleaved by the enzyme, emits a detectable signal. Exemplary substrates include chemiluminescent, fluorescent or colorimetric substrates. The presence of the signal indicates the presence of the antibody in the sample. According to a further embodiment, the intensity of the emitted signal can be correlated with the amount of the antibody in the sample.

The secondary antibody can also comprise a label such as a fluorescent label. Exemplary fluorescent labels include, but are not limited to, Fluorescein, Rhodamine, p-hydroxyphenylacetic acid, and 3-(p-hydroxyphenyl)propionic acid. After the secondary antibody is contacted with the surface of the solid support, the presence of the fluorescent signal indicates the presence of the antibody in the sample. According to a further embodiment, the intensity of the emitted signal can be correlated with the amount of the antibody in the sample.

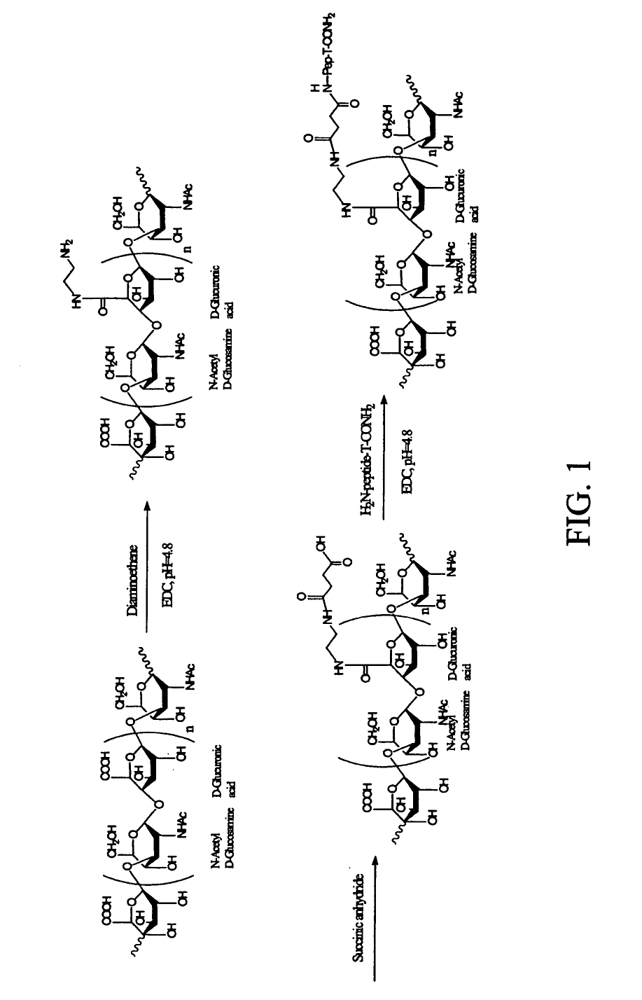

A method of making a polypeptide bioconjugate is also provided wherein a polypeptide having a free amino group is covalently linked to a polysaccharide. This method is illustrated in FIG. 1 which illustrates the synthesis of a polypeptide bioconjugate according to a second embodiment of the invention. The polysaccharide is first reacted with a diamine such that an amino group of the diamine reacts with a carboxylic acid group of the polysaccharide. The resulting product is then reacted with an acid anhydride or a dicarboxylic acid such that an anhydride group of the acid anhydride or a carboxylic acid group of the dicarboxylic acid reacts with the remaining amino group of the diamine. The polypeptide comprising a free amino group is then reacted with the resulting reaction product such that the free amino group reacts with the remaining carboxylic acid group of the dicarboxylic acid to form the polypeptide bioconjugate. Exemplary diamines include, but are not limited to, alkane diamines such as diaminoethane. Exemplary acid anhydrides include, but are not limited to, succinic anhydride and glutaric anhydride. Exemplary dicarboxylic acids include, but are not limited to, succinic acid, glutaric acid and adipic acid. Some of the steps in the reaction can be conducted under weak acidic conditions (e.g., at a pH of about 5) in the presence of EDC.

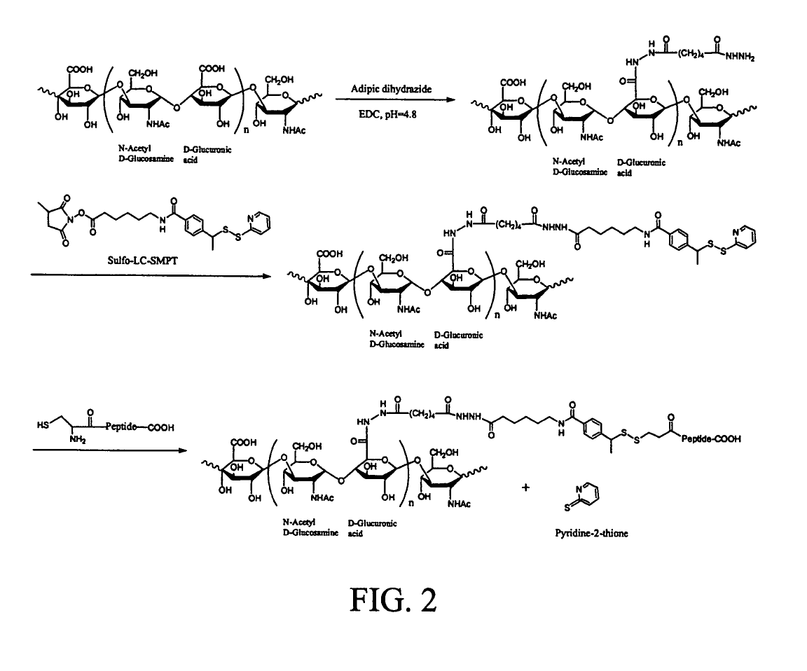

A method of making a polypeptide bioconjugate is also provided wherein a polypeptide having a free sulfhydryl (i.e., cystein) group is covalently linked to a polysaccharide. This method is illustrated in FIG. 2 which shows the synthesis of a polypeptide bioconjugate according to a third embodiment of the invention. According to this aspect of the invention, the polysaccharide is reacted with a dihydrazide such that a hydrazide group of the dihydrazide reacts with a carboxylic acid group of the polysaccharide. This step in the reaction can be conducted under weak acidic conditions (e.g., at a pH of about 5) in the presence of EDC. The resulting reaction product is then reacted with a linker compound comprising an amino reactive functional group and a thiol reactive functional group (e.g., sulfo-LC-SMPT) such that the amino reactive group of the linker compound reacts with the remaining hydrazide group of the dihydrazide. The polypeptide comprising a sulfhydryl group is then reacted with the resulting reaction product such that the free sulfhydryl group reacts with the remaining thiol reactive group of the linker compound residue to form the polypeptide bioconjugate. Exemplary dihydrazides include, but are not limited to, adipic dihydrazide, succinic dihydrazide and glutaric dihydrazide. Exemplary linker compounds comprising an amino reactive functional group and a thiol reactive functional group include, but are not limited to, sulfo-LC-SMPT (i.e., sulfosuccinimidyl-6-[α-methyl-α-(2-pyridyldithio)toluamido]hexanoate), LC-SMPT, SPDP (i.e., N-succinimidyl 3-(2-pyridyldithio)propionate) and SMCC (N-succinimidyl-4-(N-maleimidomethyl)cyclohexane-1-carboxylate).

The polysaccharride which is conjugated to the polypeptide can be hyaluronic acid (HA). The general structure of HA is shown below:

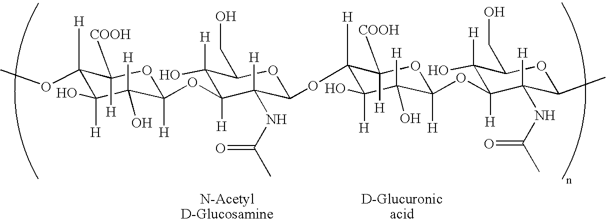

As used herein, the term “hyaluronic acid” (HA), also known as hyaluronan, includes naturally occurring forms such as those found in skin or other tissues of animals, including humans. Naturally occurring HA is typically a high molecular weight (e.g., 50,000-6,000,000 daltons) nonsulfated glycosaminoglycan (GAG) composed of repeating units of D-glucuronic acid-N-acetyl-D-glucosamine.

Various forms of HA are known in the art and/or commercially available. The HA used in the bioconjugate can be derived from a variety of sources and may be obtained as a salt. Sources of HA include human umbilical cord, rooster comb, streptococcus species, vitreous gel, and synthetic and recombinant preparations (Biochem. Biophys. Acta. 1380:377-388 (1998); U.S. Pat. No. 6,090,596). U.S. Pat. Nos. 4,141,973; 4,141,973; and 5,166,331 teach additional sources and processes of the manufacture and recovery of HA polymer analogues. Any of these sources and methods can be used to obtain HA.

The molecular weight of HA fractions may be varied using methods well known in the art. For example, low molecular weight HA may be obtained from synthetic sources or the degradation of high molecular weight compositions by heat treatment (10 minutes at 100° C.), or enzymatic digestion with hyaluronidase and size exclusion chromatography (Camenisch and McDonald, Am. J. Respir. Cell Mol. Biol. 23:431-433 (2000)).

The hyaluronic acid used in the invention may further include a core glycan, such as Gal-Gal-Xyl repeats, and/or linkage to a protein core such as N- and O-linked glycoproteins found in naturally occurring proteoglycans. It is understood in the art that an HA polymer that includes a protein core will be depleted of such protein cores when administered in vivo.

Hyaluronic Acid (HA) Polymer Analogues may also be used in the bioconjugates. The term “hyaluronic acid polymer analogue” as used herein includes hyaluronic acid, as defined herein, as well as any linear or branched glycosaminoglycan polymer comprising at least two, and preferably at least ten, repeating disaccharide units. The repeating disaccharide units include the sugar acids naturally found in linear polymeric forms of hyaluronic acid (HA), amylose, dextran and chitosan or in branched HA, amylopectin and hemi-cellulose, as well as those in chondritic sulfate, dermatan sulfate, heparan sulfate, keratan sulfate, and derivatives thereof.

The hyaluronic acid polymer analogue used in the bioconjugates is capable of forming or can be modified to form at least one covalent bond with a polypeptide.

The basic chemical structure of the HA polymer analogues of the present invention comprise repeating dissacharide sugar acids with the formula (A−B)ₙ, wherein n is at least 2 and preferably at least about 4 to about 16, more preferably at least about 4 to about 10. For instance, HA per se is comprised of alternating copoly(beta-glucuronic acid-[1->3]-beta-N-acetylglucosamine[1->4] or, according to the above formula wherein A is abbreviated as “glucuronic acid” (GlcA) and B is abbreviated as “N-acetylglucosamine” (GlcNAc), and n is at least 2, preferably at least about 4, more preferably at least about 10, and more preferably still at least about 16.

Irrespective of source, the basic chemical disaccharide structure of HA polymer analogues is functionally equivalent and in some instances (e.g., HA per se) identical to HA. Functionally equivalent HA polymer analogues may comprise the following sugar acid residue combinations: A=GlcA with B=GalNAc (chondroitin sulfate), A=GlcA or IdA (iduronic acid) and B=GalNAc (dermatan sulfate), A=GlcA or IdA and B=GlcNAc (heparan sulfate) and A=Gal and B=GlcNAc, where Gal represents a galactose, GlcN represents a glucosamine. HA polymer analogues of the invention also include the following synthetic polymers: polyphosphazines, poly(vinyl alcohols), polyamides, polycarbonates, polyvinyl ethers, polyvinyl esters, polyvinyl halides, polyvinylpyrrolidone, polyglycolides, polysiloxanes, polyurethanes, and synthetically modified natural polymers such as alkyl celluloses, hydroxyalkyl celluloses, cellulose ethers, cellulose esters, and nitrocelluloses.

HA polymer analogues of the present invention may be further derivatized to alter surface charge or functional groups for covalent linkage to a peptide. For example, the native form of HA lacks sulfates but may be derivatized to include sulfates, or hydroxyl and amino groups and the like, for covalent linkage to peptides of the present invention. HA may also be modified with sulfates to alter the charge of the polymer.

The polysaccharide is preferably a hyaluronic acid or a hyaluronic acid analogue. Although polysaccharides such as HA are described above, other biopolymers which are water soluble and biodegradable can also be used to form a bioconjugate.

According to a further embodiment, the polypeptide conjugated to the polysaccharide can comprise an antigenic determinant of an antibody whose presence in a sample is associated with a disease (e.g., a virus). For example, the polypeptide can comprise an antigenic determinant of the HIV or SARS virus. According to one embodiment, the polypeptide is an antigenic determinant of the SARS virus. Exemplary polypeptides which can be conjugated to the polysaccharide include polypeptides comprising antigenic determinants of the SARS virus as disclosed in U.S. Patent Application Ser. No. 60/475,486, filed Jun. 4, 2003, which is incorporated herein by reference in its entirety. According to one embodiment, the polypeptide conjugated to the polysaccharide comprises all or part of the following polypeptide sequence:

FERDISNVPFSPDGKPC SEQ ID NO:2

According to further embodiments, the polypeptide conjugated to the polysaccharide can comprise a peptide sequence selected from the group consisting of:

| | | |
|---|---|---|
| Peptide #78 | PDGKPC; | SEQ ID NO:23 |
| Peptide #79 | SPDGKPC; | SEQ ID NO:24 |
| Peptide #80 | FSPDGKPC; | SEQ ID NO:25 |
| Peptide #81 | PFSPDGKPC; | SEQ ID NO:26 |
| Peptide #82 | VPFSPDGKPC; | SEQ ID NO:27 |
| Peptide #83 | NVPFSPDGKPC; | SEQ ID NO:28 |
| Peptide #84 | SNVPFSPDGKPC; | SEQ ID NO:29 |
| Peptide #85 | ISNVPFSPDGKPC; | SEQ ID NO:30 |
| Peptide #86 | DISNVPFSPDGKPC; | SEQ ID NO:31 |
| Peptide #87 | RDISNVPFSPDGKPC; and | SEQ ID NO:32 |
| Peptide #88 | ERDISNVPFSPDGKPC. | SEQ ID NO:33 |

In addition to being a diagnostic agent, bioconjugates as described herein may be used as a therapeutic which, when administered to a patient (e.g., via injection), can stimulate an immune response. In particular, patients suspected of being SARS patients (e.g., patients exhibiting SARS symptoms, but not confirmed through PCR or other methods) may benefit from prompt inoculation with a bioconjugates as described herein to assist the body in the rapid generation of therapeutic titres of the SARS antibody raised against the SARS viral protein related peptides.

Bioconjugates as described herein can also be used to augment supplies of antibodies to disease-associated antigens (e.g., viral proteins such as SARS viral proteins). For example, a conventional column can be packed with a solid support (e.g., beads) wherein surfaces of the solid support comprise a bioconjugate as described herein attached thereto. The column can, for example, be used to “pull” antibodies present in the serum of recovered patients out of solution. The purified antibodies can be subsequently released from the column, collected, and administered. Isolation of the antibodies in this method also permits wider patient selection, long-term storage of the antibody, transportation of the antibody to distant sites, etc. Thus, bioconjugates as described herein can be used both to augment short-term solutions and to provide long-term solutions that include diagnosis of SARS infection, therapeutic treatment of SARS-infected patients, and vaccination of patients.

### EXAMPLES AND PREPARATIONS

### Synthesis of Hyloranate Derivatives for Peptide Conjugation

Sodium hyloranate 80 mg (Mw 600,000-1,000,000) was dissolved in 0.1 M MES buffer at pH 5.0. To this solution was added diaminomethane 100 mg and EDC 100 mg. The reaction mixture was kept overnight at room temperature. The excess amount of starting materials was removed by dialysis in deionized water for 6 hours using dialysis tubing with MWCO=12 K-14 K. The liquid was lyophilized and offer white fiber-like solid. Ninhydrin Assay confirmed that the primary amino groups were present in the product.

Ethylenediamino hyloranate 80 mg was dissolved in 0.1 M phosphate buffer at pH 7.5. To this solution was added succinic anhydride 80 mg. The reaction mixture was rotated at room temperature for 4 hours. The excess amount of starting materials was removed by dialysis in deionized water for 6 hours using dialysis tube with MWCO=12K-14K. The liquid was lyophilized and the reaction product had the appearance of a white fiber-like solid.

### Synthesis of Peptide-Hyloranate Conjugate

Dissolve ethylenediamino hyloranate in 0.1 N MES buffer at pH 5.0. To this solution added a solution of EDC 12 μl at 10 mg/mL and peptide-T amide in DMF at 10 mg/mL. Peptide-T amide has an amino acid sequence as set forth below:

| | |
|---|---|
| ASTTTNYTCONH₂ | SEQ ID NO:1 |

The mixture was stirred at room temperature for 3 hours. The starting material and byproducts were removed using dialysis tube with MWCO=12-14K. The liquid was lyophilized and the reaction product had the appearance of a white solid.

### Immuno-Dot-Blot Procedure on HA-L-PT

HA-L-PT or free PT was dissolved in PBS (1 mg/ml) and 10 μl of each peptide was blotted onto the Immunobilon-nitrocellular membrane (Millipore, Billerica Mass.). After the membrane was air-dried, it was blocked with 5% non-fat dry milk in PBS for 60 minutes on a shaker. The membrane was then washed three times for 15 minutes each with TPBS (PBS with 0.1% Tween-20) on the shaker. HA-L-PT and free PT loaded membrane was incubated with peptide T rabbit antiserum (1:250 in PBS containing 3% non-fat dry milk) in a plastic container at room temperature for 2 hours on a shaker. The membrane was washed three times for fifteen (15) minutes each with TPBS (PBS with 0.1% Tween-20) with continuous agitation. Phosphatase labeled goat anti-rabbit antibody (1:2000 dilution in PBS containing 3% nonfat dry milk) was used as a secondary antibody to detect peptide T specific antibodies. After incubation at room temperature for 1 hour, the membrane was washed twice for 15 minutes each with TPBS. The rabbit immunoglobulin against peptide T was detected with the BCIP/NBT liquid substrate.

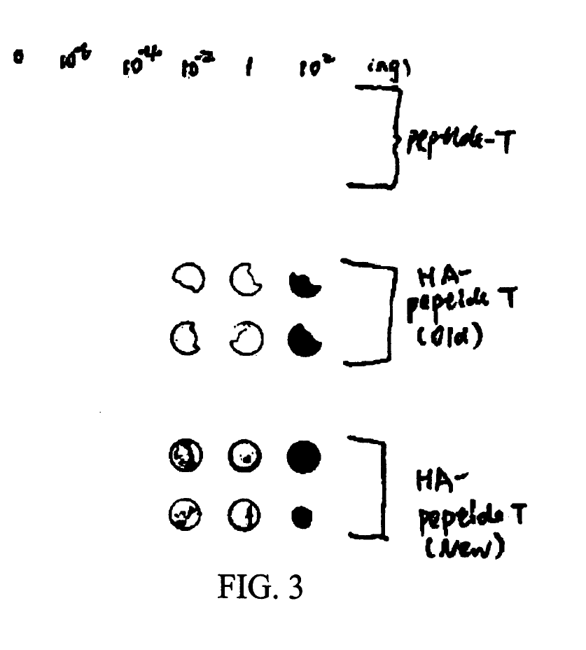

FIG. 3 is an image of the membrane after the color was developed. The amount in nanograms (ng) of analyte at each location on the membrane is shown at the top of the figure. As can be seen from FIG. 3, conjugation of the peptide to hyaluronic acid significantly improves the sensitivity of the assay allowing for the detection of the polypeptide in amounts of as little as 10⁻² ng whereas equivalent or greater amounts of non-conjugated polypeptide showed no detectable signal. In particular, no detectable signal was observed for any amount of non-conjugated polypeptide tested, including amounts of 10⁻², 1 or 10² ng, at which levels detectable signals were observed with HA-polypeptide conjugate.

ELISA Procedure for Testing HA-Peptide T (I)

100 μl of Serial dilutions of HA-Peptide T (HA-L-PT), or free Peptide T (PT) were coated in each well of microplate in coating buffer (NaHCO₃/Na₂CO₃, 563/215 mM, pH 9.6). The range of the peptide conjugate or free peptide was from 100 μg/ml to 10 fg/ml. Each dilution of peptide HA conjugate or free peptide was assayed in duplicate. The plate was kept at 4° C. for overnight and then washed for 3 times with wash buffer (Tris buffered saline, TBS, pH 7.2, supplemented with 0.2% Tween 20). After blotting, the plate was blocked with 150 μl of a mixture of 1% BSA and 1% dry milk in TBS at 37° C. for 30 min. The plate was washed again for 5 times and 100 μl of diluted (1:500 in 0.1% BSA in TBS) rabbit antiserum against peptide T was added to each well of the plate. The plate was incubated at 37° C. for 90 min. At the end of incubation, the plate was washed for 5 times and 100 μl of diluted (1:1000 in 0.1% BSA in TBS) goat anti-rabbit alkaline phosphatase conjugate was added to each well. The plate was incubated at 37° C. for 30 min. After the final wash (5 times in wash buffer), the plate was added with 100 μl of p-nitro phenyl phosphate substrate solution (2 mg/ml in substrate buffer containing 16.2 mM NaHCO₃, 17.0 mM Na₂CO₃ and 1.0 mM MgCl₂6H₂O) and incubated for additional 30 min at room temperature. Optical density (OD) was measured at 405 nm.

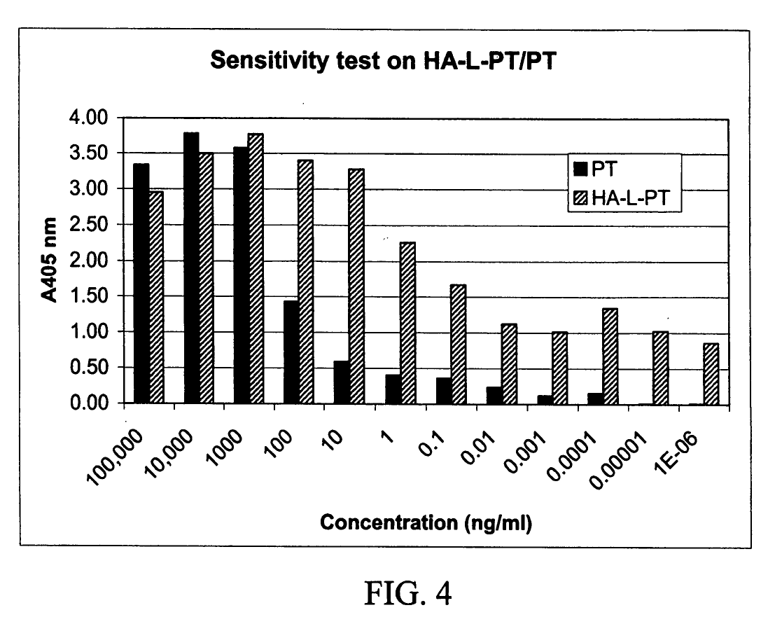

FIG. 4 shows optical density at 405 nm as a function of concentration in ng/ml of peptide T (PT) and a HL-L-PT conjugate. As can be seen from FIG. 4, the sensitivity of the assay was significantly higher using the Peptide T conjugate than with the non-conjugated peptide T. The difference in sensitivity was particularly pronounced at lower concentrations (i.e., at concentrations of 100 ng/ml or less).

### ELISA Procedure for Testing HA-Peptide T (II)

100 μl of HA-Peptide T (10 pg/ml), or free Peptide T (10 pg/ml) were coated in each well of microplate in coating buffer (NaHCO₃/Na₂CO₃, 563/215 mM, pH 9.6). The plate was kept at 4° C. for overnight and then washed for 3 times with wash buffer (Tris buffered saline, TBS, pH 7.2, supplemented with 0.2% Tween 20). After blotting, the plate was blocked with 150 μl of a mixture of 1% BSA and 1% dry milk in TBS at 37° C. for 30 min. The plate was washed again for 5 times and 100 μl of each diluted (1:100, 1:1000, 1:10000 and 1:100000 in 0.1% BSA in TBS) rabbit antiserum against peptide T was added to each well of the plate. Each dilution of peptide HA conjugate or free peptide was assayed in duplicate. The plate was incubated at 37° C. for 90 min. At the end of incubation, the plate was washed for 5 times and 100 μl of diluted (1:1000 in 0.1% BSA in TBS) goat anti-rabbit alkaline phosphatase conjugate was added to each well. The plate was incubated at 37° C. for 30 min. After the final wash (5 times in wash buffer), the plate was added with 100 μl of p-nitro phenyl phosphate substrate solution (2 mg/ml in substrate buffer containing 16.2 mM NaHCO₃, 17.0 mM Na₂CO₃ and 1.0 mM MgCl₂6H₂O) and incubated for additional 30 min at room temperature. Optical density (OD) was measured at 405 nm.

The results indicated that peptide T antibodies can be detected at a dilution of 1:10,000 when using the HA-Peptide T conjugate (i.e., at a concentration of 10 pg/ml coated on an ELISA plate). There was no significant difference detected among all antiserum dilutions when using non HA conjugated peptide T under the same conditions.

### Synthesis of Hyloranate Conjugate

Sodium hyloranate 169 mg (Mw 600,000-1,000,000) was dissolved in 0.1 M MES buffer at pH 4.8. To this solution was added Adipic dihydrazide 78 mg and EDC 170 mg. The reaction mixture was kept at room temperature for 4 hours. The excess amount of starting materials and byproduct were removed by dialysis in deionized water for 6 hours using dialysis tubing with MWCO=12K-14K. The liquid was lyophilized and offer white fiber-like solid. Ninhydrin Assay confirmed that the primary amino groups were present in the product.

Adipic dihydrazide hyloranate 50 mg was dissolved in 0.1 M phosphate buffer at pH 7.4. To this solution was added a solution of sulfosuccinimidyl-6-[α-methyl-α-(2-pyridyldithio)toluamido]hexanoate (sulfo-LC-SMPT) 80 mg in 0.5 mL of DMF and 1.0 mL of phosphate buffer. The reaction mixture was put on a shaker at room temperature for 3 hours. The reaction mixture was purified by dialysis in deionized water for 6 hours using dialysis tube with MWCO=12K-14K. The liquid was lyophilized and the reaction product was a white solid of hyaluronic 6-[α-methyl-α-(2-pyridyldithio)toluamido]hexanoate.

### Synthesis of Peptide Library

A library of synthetic peptides was synthesized using well-established Fmoc method on solid support. Cystein residue was attached on the N-terminal of the targeting peptides. The crude peptides were obtained after cleaving the peptides from solid support and deblocking the protection group. The crude peptide could be purified by C18-HPLC if needed.

### Synthesis of Hyloranate Peptide Conjugate Library

Hyaluronic 6-[α-methyl-α-(2-pyridyldithio)toluamido]-hexanoate was dissolved in 50 mL of phosphate buffer pH 7.4 at 1 mg/ml. Load 100 μl of this solution in each well of a 96-well plate. Add at least 2-fold excess amount of 96 peptide solutions from the pre-synthesized peptide library into the 96-well plate. The 96-well plate was rocked at room temperature for 2 hours. The process of the reaction was monitored using 96-well spectrophotometer for the release of pyridine-2-thion, which has characteristic absorption at 344 nm. The reaction should be stopped when two reading are very close at 15 minutes interval. The crude reaction mixture can be used directly without further purification. The loading amount of peptide on hyloranate carrier was adjusted through varying the amount of peptide solution. The peptide load range is from 2% to 100% based on the application.

### Conjugation of Peptide-26 with Modified Hyloranate

As a specific example, hyaluronic 6-[α-methyl-α-(2-pyridyldithio) toluamido]-hexanoate 0.9 mg was dissolved in PBS buffer pH 7.4 at 1 mg/ml. To this solution was added a DMF/buffer solution of peptide-26 (1.5 mg at 3 mg/ml). Peptide-26 has an amino acid sequence as set forth below:

| | |
|---|---|
| FERDISNVPFSPDGKPC | SEQ ID NO:2 |

Peptide-26 was originated from spike protein of SARS coroner virus. The reaction mixture was rocked for 3 hours, and monitored with spectrophtometer at 344 nm. The mixture was dialyzed for 3.5 hours against deionized water with MWCO=5000. After lyophlization, the resulting conjugate was dissolved in 2 ml of PBS buffer. Based on the calculation, the actual concentration of peptide-26 loaded on the conjugate is 0.25 mg/ml. This solution was used for ELISA assay and dot blotting assay.

### ELISA Procedure for Testing of SARS Patient Serum Using Hyloranate Peptide-26 Conjugate

A 96-well ELISA plate was coated with hyloranate peptide-26 solution at 0, 3.1 μg, 12.5 μg and 50 μg at 37 ° C. for 1 hour. Block the ELISA plate with five (5) % non-fat dry milk in PBS for 1 hour. After washing 3 times with TPBS (PBS with 0.1% Tween-20), the ELISA plate was incubated with serum samples (1:400 in PBS) at room temperature for 2 hours and washed three times for 15 minutes each with TPBS (PBS with 0.1% Tween-20). Phosphatase labeled goat anti-human IgA+IgG+IgM antibody (1:1000 dilution in PBS containing 3% nonfat dry milk) was used as secondary antibody to detect peptide specific antibodies. After incubation at room temperature for I hour, the plate was washed twice for 15 minutes each with TPBS. The human immunoglobulin specific peptides were detected with p-nitrophenyl phosphate substrate at 405 nm.

TABLE 1
ELISA Test of SARS Patient Serum vs Normal Person Using
Hyloranate Peptide-26 (15 min.)

| Peptide-26 conjugate | A405 (SARS patient) | A405 (normal person) |
|---|---|---|
| 0 μg | 0.105 | 0.12 |
| 0 μg | 0.092 | 0.12 |
| 3.1 μg | 0.648 | 0.09 |
| 3.1 μg | 0.697 | 0.09 |
| 12.5 μg | 0.738 | 0.13 |
| 12.5 μg | 0.718 | 0.11 |
| 50.0 μg | 0.67 | 0.11 |
| 50.0 μg | 0.74 | 0.11 |

TABLE 2
ELISA Test of SARS Patient Serum vs Normal Person Using
Hyloranate Peptide-26 (30 min.)

| Peptide-26 conjugate | A405 (SARS patient) | A405 (normal person) |
|---|---|---|
| 0 μg | 0.12 | 0.12 |
| 0 μg | 0.11 | 0.13 |
| 3.1 μg | 1.23 | 0.11 |
| 3.1 μg | 1.23 | 0.11 |
| 12.5 μg | 1.19 | 0.15 |
| 12.5 μg | 1.21 | 0.14 |
| 50.0 μg | 1.23 | 0.14 |
| 50.0 μg | 1.26 | 0.14 |

The testing results show the strong specificity of peptide-26 toward SARS patient antibody. Therefore, peptide-26 conjugates can be used for both diagnostic and vaccine applications.

### Synthesis of Hyloranate Multiple-Peptide Conjugate

Hyaluronic 6-[α-methyl-α-(2-pyridyldithio)toluamido]-hexanoate was dissolved in phosphate buffer pH 7.4 at 1 mg/ml. Load 500 μl of this solution in eppindorf vial. Add a solution of 12 peptides from the pre-synthesized peptide library into the vial. The vial was rocked at room temperature for 2 hours. The process of the reaction was monitored using 96-well spectrophotometer for the release of pyridine-2-thion, which has characteristic absorption at 344 nm. The crude reaction mixture can be used directly without further purification. The loading amount of peptide on hyloranate carrier was adjusted through varying the amount of free peptide solution. The peptide load range is from 2% to 100% based on the application.

In addition to Sulfo-LC-SMPT, the linker may also include LC-SMPT, SMPT (Pierce), SPDP (N-succinimidyl 3-(2-pyridyldithio)propionate) and SMCC (N-succinimidyl-4-(N-maleimidomethyl)cyclohexane-1-carboxylate. However, SMPT linker is preferred due to its better stability in vitro and in vivo than the SPDP and SMCC linkers.

### Immobilization of Hyloranate Peptide Conjugate on Nitrocellulose Membrane

All hyloranate peptide conjugates were coated onto a rectangular 96-well plate nitrocellulose membrane, a 96-well Minifold Dot Blot Plate. Human IgG was used as a positive control. The membrane was allowed air dry and then blocked with 10 mM sodium phosphate (pH 7.5)/150 mM NaCl (PBS) containing 5% nonfat dry milk for one hour at room temperature. The membrane was washed twice for 15 minutes each with PBS and then allowed air dry.

### Detection of SARS Specific Peptides

The resulting affixed membrane was exposed to 56 serum samples. In particular, the membrane was incubated with the serum samples (1:400 in PBS containing 3% nonfat dry milk) at room temperature for 2 hours and washed three times for 15 minutes each with TPBS (PBS with 0.1% Tween-20). Phosphatase labeled goat anti-human IgA+IgG+IgM antibody (1:2000 dilution in PBS containing 3% nonfat dry milk) was used as secondary antibody to detect peptide specific antibodies. After incubated at room temperature for 1 hour, the membrane was washed twice for 15 minutes each with TPBS. The human immunoglobulin specific peptides were detected with BCIP/NBT substrate.

Using this method, the sera from 46 well documented SARS patients and the sera from 10 normal people were tested. As a result of this testing, twenty SARS viral specific antibody-binding peptides have been identified from 40 serum samples out of 46 SARS patient samples. These peptides are listed below:

| | |
|---|---|
| FKCYGVSATKLNDL | SEQ ID NO:3 |
| SKRFQPFQQFGRDVSDFTD | SEQ ID NO:4 |
| AEHVDTSYECDIP | SEQ ID NO:5 |
| REVFAQVKQMYKTPTLKYF | SEQ ID NO:6 |
| LLTDDMIAAY | SEQ ID NO:7 |
| RSDTLYLTQDLFLPFYSNVTG | SEQ ID NO:8 |
| ASSEVAVLYQDVNCTDVSTAIHADQLTPAWR | SEQ ID NO:9 |
| AISSVLNDILSRLDKVEAEVQIDR | SEQ ID NO:10 |
| GGRNGARPKQRRPQG | SEQ ID NO:11 |
| LTQHGKEELRFPRGQG | SEQ ID NO:12 |
| ATRRVRGGDGKMKELSPRWY | SEQ ID NO:13 |
| SQASSRSSSRSRGNSRNSTP | SEQ ID NO:14 |
| ALLLLDRLNQLESKVSGKGQ | SEQ ID NO:15 |
| TVTKKSAAEASKKPRQKRTATKQYN | SEQ ID NO:16 |
| TYHGAIKLDDKDPQFKDNVI | SEQ ID NO:17 |
| PTEPKKDKKKKTDEAQP | SEQ ID NO:18 |
| LPQRQKKQPTVTLLP | SEQ ID NO:19 |
| PAADMDDFSRQLQNSMSGASADST | SEQ ID NO:20 |
| PAADMDDFSRQLQNS | SEQ ID NO:21 |
| FPSVYAW | SEQ ID NO:22 |

These polypeptides are disclosed in provisional U.S. patent application Ser. No. 60/475,486, filed Jun. 4, 2003 and provisional U.S. patent application Ser. No. 60/524,614. Any of the polypeptides disclosed in the aforementioned applications can be conjugated to a polysaccharide to form a bioconjugate for use in an assay.

Membrane strips were coated with different mixtures of HA conjugated SARS polypeptides. Six (6) dots and a positive control were used on each strip. Each mixture of conjugates was duplicated once (i.e., two dots for each mixture of conjugates). The three groups of SARS antigenic HA-peptide conjugate mixtures are designated X₁, X₂ and X₃. The conjugates were synthesized based on the methods disclosed herein. Mixture X₁, is a mixture of 14 HA-peptide conjugates containing 14 different polypeptides having the amino acid sequences of SEQ ID NOS: 2, 3, 4, 5, 6, 7, 9, 11, 13, 15, 18, 19, 20 and 21. Mixture X₂ is a mixture of 10 HA-peptides conjugates containing 10 different polypeptides having the amino acid sequences of SEQ ID NO: 2, 3, 4, 5, 7, 9, 15, 18, 19, 21. Mixture X₃ is a mixture of 10 HA-peptide conjugates containing 10 different polypeptides having the amino acid sequences of SEQ ID NO: 2, 3, 4, 5, 9, 13, 15, 17, 18 and 21.

These strips were tested against various dilutions of normal serum or SARS patient serum. The serum dilutions employed were 1:400, 1:800, 1:1600 and 1:3200. The tests conducted on the SARS patient serum were positive at all dilutions tested and for all conjugate mixtures.

As described above, a plurality of bioconjugates each comprising a polysaccharide and a polypeptide can be disposed in discrete regions on the surface of a solid support. The bioconjugates can be disposed on the solid support surface in the form of an array (e.g., a microarray). The plurality of bioconjugates may comprise a plurality of different bioconjugates (e.g., the polypeptides in the different bioconjugates may have different amino acid sequences). The array may be used in a method for screening a plurality of different polypeptides for their ability to interact with a component of a sample. An exemplary method of screening comprises: depositing a sample on an array of conjugates of the polypeptides to be screened and detecting the interaction of the component in the sample with the immobilized polypeptide conjugates in each region on the support surface. The component in the sample may be a protein (e.g., an antibody).

Although antigen/antibody interactions are described above, various other interactions can be detected including, but not limited to, antibody/hapten, enzyme/substrate, carrier protein/substrate, receptor/hormone, receptor/effector, protein/DNA, protein/RNA and repressor/inducer. The interaction being screened may involve binding and/or catalysis. The assayed interaction may be between a potential drug candidate and a plurality of potential drug targets. For instance, a synthesized organic compound may be tested for its ability to act as an inhibitor to a plurality of different receptor/polysaccharide conjugates.

Another aspect of the invention provides for a method for screening a plurality of polypeptides for their ability to bind a particular component of a sample. This method comprises depositing the sample on an array comprising conjugates of the polypeptides to be screened and detecting, either directly or indirectly, for the presence or amount of the particular component retained in each region of the array. According to a preferred embodiment, the method further comprises the intermediate step of washing the array to remove any unbound or nonspecifically bound components of the sample from the array before the detection step. In another embodiment, the method further comprises the additional step of characterizing the particular component retained on at least one region of the array. The particular component may optionally be a protein.

The step of characterizing the particular component retained on a region of the array can be used to identify the nature of the component bound to the polypeptide in a particular region of the array. In some cases, the identity of the component may not be known and the purpose of the characterization may be the initial identification of the mass, sequence, structure and/or activity (if any) of the bound component. In other cases, the basic identity of the component may be known, but some information about the component may not be known. For instance it may be known that the component is a particular protein, but the post-translational modification, activation state, or some other feature of the protein may not be known.

In another embodiment of the invention, a method of assaying for protein-protein binding interactions is provided which comprises: first, depositing a sample comprising at least one protein to be assayed for binding to the polypeptide conjugate array of the invention; and then detecting, either directly, or indirectly, for the presence or amount of the protein from the sample which is retained at each region of the array. In a preferred embodiment, the method further comprises an additional step prior to the detection step which comprises washing the array to remove unbound or nonspecifically bound components of the sample from the array.

Another embodiment provides a method of assaying in parallel for the presence of a plurality of analytes in a sample which can react with one or more of the immobilized polypeptide conjugates of the array. This method comprises depositing the sample on the array and detecting the interaction of the analyte with the immobilized polypeptide conjugate in each region.

In still another embodiment of the invention, a method of assaying in parallel for the presence of a plurality of analytes in a sample which can bind one or more of the immobilized polypeptides of the array is provided which comprises depositing the sample on the surface of the array and detecting, either directly or indirectly, for the presence or amount of analyte retained in each region of the array. In a preferred embodiment, the method further comprises the step of washing the array to remove any unbound or non-specifically bound components of the sample from the array prior to detection.

The array may be used in a diagnostic manner wherein the plurality of analytes being assayed are indicative of a disease condition or the presence of a pathogen in an organism. In such embodiments, the sample which is delivered to the array will then typically be derived from a body fluid or a cellular extract from the organism.

The array may be used for drug screening wherein a potential drug candidate is screened directly for its ability to bind or otherwise interact with an array of polypeptide conjugates. Alternatively, a plurality of potential drug candidates may be screened in parallel for their ability to bind or otherwise interact with one or more immobilized polypeptide conjugates in an array. The drug screening process may optionally involve assaying for the interaction, such as binding, of at least one analyte or component of a sample with one or more immobilized polypeptide conjugates in an array, both in the presence and absence of the potential drug candidate. In this manner, the potential drug candidate can be tested for its ability to act as an inhibitor of the interaction or interactions being assayed.

While the foregoing specification teaches the principles of the present invention, with examples provided for the purpose of illustration, it will be appreciated by one skilled in the art from reading this disclosure that various changes in form and detail can be made without departing from the true scope of the invention.

1. A method for detecting the presence or amount of an analyte in a sample comprising:
contacting a surface of a solid support with the sample; and
detecting analyte on the surface of the solid support;
wherein the surface of the solid support comprises a bioconjugate of a polypeptide and a polysaccharide and wherein the polypeptide of the bioconjugate is capable of interacting with the analyte.

2. The method of claim 1, wherein the polypeptide of the bioconjugate is capable of binding to the analyte.

3. The method of claim 1, wherein the analyte is selected from the group consisting of an antibody, an immunoglobulin, an enzyme, and a hormone.

4. The method of claim 1, wherein the analyte is an antibody and wherein the polypeptide comprises an antigenic determinant of the antibody.

5. The method of claim 1, wherein the solid support is a membrane or an assay plate.

6. The method of claim 1, wherein the solid support is an ELISA plate.

7. The method of claim 4, wherein the polypeptide comprises the amino acid sequence of peptide-T (SEQ ID NO:1) or peptide-26 (SEQ ID NO:2).

8. The method of claim 1, wherein the polysaccharide is a hyaluronic acid or a hyaluronic acid analogue.

9. The method of claim 4, wherein detecting comprises:
contacting the surface of the solid support with a secondary antibody which binds to the analyte; and
detecting the secondary antibody.

10. The method of claim 9, wherein the secondary antibody is labeled.

11. The method of claim 10, wherein the secondary antibody is labeled with an enzyme label or a fluorescent label.

12. The method of claim 1, wherein a plurality of bioconjugates each comprising a polypeptide having a different amino acid sequence are deposited in different discrete regions on the surface of the solid support.

13. The method of claim 1, further comprising:
washing the solid support surface after contact of the solid support surface with the sample and before contact of the solid support surface with the secondary antibody.

14. The method of claim 1, further comprising:
washing the solid support surface after contact of the solid support surface with the secondary antibody and before detection; and/or
washing the solid support surface after depositing the bioconjugate on the solid support and before contact with the sample.

15. A method of making a polypeptide bioconjugate comprising:
reacting a polysaccharide with a first linker compound having at least two carboxylic acid reactive groups such that one carboxylic acid reactive group of the first linker compound reacts with a carboxylic acid group of the polysaccharide;
reacting the resulting reaction product with a dicarboxylic acid such that a carboxylic acid group of the dicarboxylic acid reacts with the remaining carboxylic acid reactive group of the first linker compound; and
reacting a polypeptide comprising a free amino group with the resulting reaction product such that the free amino group reacts with the remaining carboxylic acid group of the dicarboxylic acid residue to form the polypeptide bioconjugate.

16. The method of claim 15, wherein the first linker compound is a diamine.

17. The method of claim 16, wherein the diamine is an alkane diamine.

18. The method of claim 16, wherein the diamine is diaminoethane.

19. The method of claim 15, wherein the dicarboxylic acid is succinic anhydride.

20. The method of claim 15, wherein each reaction is conducted under acidic conditions.

21. The method of claim 15, wherein each reaction is conducted at a pH of 4.5 to 7.

22. The method of claim 15, wherein each reaction is conducted at a pH of about 5.

23. The method of claim 21, wherein each reaction is conducted in the presence of EDC.

24. The method of claim 15, wherein the polypeptide is peptide-T amide (SEQ ID NO:1).

25. The method of claim 15, wherein the polysaccharide is a hyaluronic acid or a hyaluronic acid analogue.

26. A bioconjugate made by a method as set forth in claim 15.

27. A bioconjugate comprising a polysaccharide covalently linked to a polypeptide, wherein the polypeptide comprises the amino acid sequence of one of SEQ ID NOS:2-22.

28. The bioconjugate of claim 27, wherein the polypeptide comprises the amino acid sequence of peptide-T (SEQ ID NO:1) or peptide-26 (SEQ ID NO:2).

29. The bioconjugate of claim 27, wherein the polysaccharide is a hyaluronic acid or a hyaluronic acid analogue.

30. An article of manufacture comprising a bioconjugate as set forth in claim 26 disposed on a surface of a solid support.

31. The article of manufacture of claim 27, wherein the solid support is a membrane or an assay plate.

32. The article of manufacture of claim 27, wherein the solid support is an ELISA plate.

33. A method of making a polypeptide bioconjugate comprising:
reacting a polysaccharide with a dihydrazide such that a hydrazide group of the dihydrazide reacts with a carboxylic acid group of the polysaccharide to form a first reaction product;
reacting the first reaction product with a linker compound comprising an amino reactive functional group and a thiol reactive functional group such that the amino reactive group of the linker compound reacts with the remaining hydrazide group of the dihydrazide to form a second reaction product;
reacting a polypeptide comprising a sulfhydryl group with the second reaction product such that the free sulfhydryl group reacts with the remaining thiol reactive group of the linker compound residue to form the polypeptide bioconjugate.

34. The method of claim 33, wherein the reaction of the polysaccharide with the dihydrazide is conducted at a pH of 4.5 to 7.

35. The method of claim 33, wherein the reaction of the polysaccharide with the dihydrazide is conducted at a pH of about 5 and in the presence of EDC.

36. The method of claim 33, wherein the dihydrazides is adipic dihydrazide.

37. The method of claim 33, wherein the linker compound comprising an amino reactive functional group and a thiol reactive functional group is selected from the group consisting of sulfo-LC-SMPT, LC-SMPT, SPDP (N-succinimidyl 3-(2-pyridyldithio)propionate) and SMCC (N-succinimidyl-4-(N-maleimidomethyl)cyclohexane-1-carboxylate).

38. The method of claim 33, wherein the linker compound comprising an amino reactive functional group and a thiol reactive functional group is Sulfo-LC-SMPT.

39. A bioconjugate made by a method as set forth in claim 33.

40. An article of manufacture comprising a bioconjugate as set forth in claim 39 disposed on a surface of a solid support.

41. The article of manufacture of claim 40, wherein the solid support is a membrane or an assay plate.

42. The article of manufacture of claim 40, wherein the solid support is an ELISA plate.

43. A bioconjugate comprising a polypeptide residue conjugated to a polysaccharide residue by a linker moiety wherein the linker moiety comprises the residue of: a diamino compound; a dihydrazide compound; a reaction product of a diamino and a dicarboxylic acid compound; and a reaction product of a dihydrazide and a compound comprising an amino reactive functional group and a thiol reactive functional group.

44. An article of manufacture comprising a bioconjugate as set forth in claim 43 disposed on a surface of a solid support.

45. The article of manufacture of claim 44, wherein the solid support is a membrane or an assay plate.

46. The article of manufacture of claim 44, wherein the solid support is an ELISA plate.


SEQUENCE LISTING (33 sequences)

SEQ ID NO: 1 - protein, 8 aa, Artificial Sequence
  polypeptides comprising antigenic determinants of the SARS virus
ASTTTNYT

SEQ ID NO: 2 - protein, 17 aa, Artificial Sequence
  polypeptides comprising antigenic determinants of the SARS virus
FERDISNVPFSPDGKPC

SEQ ID NO: 3 - protein, 14 aa, Artificial Sequence
  SARS viral specific antibody-binding peptide
FKCYGVSATKLNDL

SEQ ID NO: 4 - protein, 19 aa, Artificial Sequence
  SARS viral specific antibody-binding peptide
SKRFQPFQQFGRDVSDFTD

SEQ ID NO: 5 - protein, 13 aa, Artificial Sequence
  SARS viral specific antibody-binding peptide
AEHVDTSYECDIP

SEQ ID NO: 6 - protein, 19 aa, Artificial Sequence
  SARS viral specific antibody-binding peptide
REVFAQVKQMYKTPTLKYF

SEQ ID NO: 7 - protein, 10 aa, Artificial Sequence
  SARS viral specific antibody-binding peptide
LLTDDMIAAY

SEQ ID NO: 8 - protein, 21 aa, Artificial Sequence
  SARS viral specific antibody-binding peptide
RSDTLYLTQDLFLPFYSNVTG

SEQ ID NO: 9 - protein, 31 aa, Artificial Sequence
  SARS viral specific antibody-binding peptide
ASSEVAVLYQDVNCTDVSTAIHADQLTPAWR

SEQ ID NO: 10 - protein, 24 aa, Artificial Sequence
  SARS viral specific antibody-binding peptide
AISSVLNDILSRLDKVEAEVQIDR

SEQ ID NO: 11 - protein, 15 aa, Artificial Sequence
  SARS viral specific antibody-binding peptide
GGRNGARPKQRRPQG

SEQ ID NO: 12 - protein, 16 aa, Artificial Sequence
  SARS viral specific antibody-binding peptide
LTQHGKEELRFPRGQG

SEQ ID NO: 13 - protein, 20 aa, Artificial Sequence
  SARS viral specific antibody-binding peptide
ATRRVRGGDGKMKELSPRWY

SEQ ID NO: 14 - protein, 20 aa, Artificial Sequence
  SARS viral specific antibody-binding peptide
SQASSRSSSRSRGNSRNSTP

SEQ ID NO: 15 - protein, 20 aa, Artificial Sequence
  SARS viral specific antibody-binding peptide
ALLLLDRLNQLESKVSGKGQ

SEQ ID NO: 16 - protein, 25 aa, Artificial Sequence
  SARS viral specific antibody-binding peptide
TVTKKSAAEASKKPRQKRTATKQYN

SEQ ID NO: 17 - protein, 20 aa, Artificial Sequence
  SARS viral specific antibody-binding peptide
TYHGAIKLDDKDPQFKDNVI

SEQ ID NO: 18 - protein, 17 aa, Artificial Sequence
  SARS viral specific antibody-binding peptide
PTEPKKDKKKKTDEAQP

SEQ ID NO: 19 - protein, 15 aa, Artificial Sequence
  SARS viral specific antibody-binding peptide
LPQRQKKQPTVTLLP

SEQ ID NO: 20 - protein, 24 aa, Artificial Sequence
  SARS viral specific antibody-binding peptide
PAADMDDFSRQLQNSMSGASADST

SEQ ID NO: 21 - protein, 15 aa, Artificial Sequence
  SARS viral specific antibody-binding peptide
PAADMDDFSRQLQNS

SEQ ID NO: 22 - protein, 7 aa, Artificial Sequence
  SARS viral specific antibody-binding peptide
FPSVYAW

SEQ ID NO: 23 - protein, 6 aa, Artificial Sequence
  polypeptides comprising antigenic determinants of the SARS virus
PDGKPC

SEQ ID NO: 24 - protein, 7 aa, Artificial Sequence
  polypeptides comprising antigenic determinants of the SARS virus
SPDGKPC

SEQ ID NO: 25 - protein, 8 aa, Artificial Sequence
  polypeptides comprising antigenic determinants of the SARS virus
FSPDGKPC

SEQ ID NO: 26 - protein, 9 aa, Artificial Sequence
  polypeptides comprising antigenic determinants of the SARS virus
PFSPDGKPC

SEQ ID NO: 27 - protein, 10 aa, Artificial Sequence
  polypeptides comprising antigenic determinants of the SARS virus
VPFSPDGKPC

SEQ ID NO: 28 - protein, 11 aa, Artificial Sequence
  polypeptides comprising antigenic determinants of the SARS virus
NVPFSPDGKPC

SEQ ID NO: 29 - protein, 12 aa, Artificial Sequence
  polypeptides comprising antigenic determinants of the SARS virus
SNVPFSPDGKPC

SEQ ID NO: 30 - protein, 13 aa, Artificial Sequence
  polypeptides comprising antigenic determinants of the SARS virus
ISNVPFSPDGKPC

SEQ ID NO: 31 - protein, 14 aa, Artificial Sequence
  polypeptides comprising antigenic determinants of the SARS virus
DISNVPFSPDGKPC

SEQ ID NO: 32 - protein, 15 aa, Artificial Sequence
  polypeptides comprising antigenic determinants of the SARS virus
RDISNVPFSPDGKPC

SEQ ID NO: 33 - protein, 16 aa, Artificial Sequence
  polypeptides comprising antigenic determinants of the SARS virus
ERDISNVPFSPDGKPC